(19) United States
(12) Patent Application Publication
(10) Pub. No.: US 2022/0142878 A1
(43) Pub. Date: May 12, 2022

(54) SKIN ANTI-AGEING AND REVITALIZING COSMETIC GEL COMPOSITION

(71) Applicant: CONTRAD SWISS S.A., Lugano (CH)

(72) Inventor: Joanne SERRENTINO, Sainte-Catherine-de-Hatley (CA)

(21) Appl. No.: 17/095,137

(22) Filed: Nov. 11, 2020

(51) Int. Cl. A61K 8/04; A61K 8/64; A61K 8/81; A61K 8/73; A61K 8/67; A61K 8/44; A61K 8/34; A61K 8/368; A61K 8/362; A61K 8/86; A61K 8/92; A61K 8/19; A61Q 19/08

(52) U.S. Cl. CPC A61K 8/042; A61K 2800/592; A61K 8/8141; A61K 8/73; A61K 8/673; A61K 8/44; A61K 8/345; A61K 8/368; A61K 8/362; A61K 8/86; A61K 8/922; A61K 8/19; A61Q 19/08; A61K 2800/48; A61K 2800/51; A61K 2800/524; A61K 8/64

(57) ABSTRACT

A water-based cosmetic gel composition for topical application to the skin, having an anti-ageing, moisturising, brightening and revitalizing effect on the skin, is provided. The water-based cosmetic gel composition includes a combination of biomimetic peptides and, optionally, hyaluronic acid salt, panthenol, and one or more ingredients selected from the group consisting of thickening agents, wetting agents, solubilizing agents, chelating agents, pH adjusting agents, preserving agents, and combinations thereof.

### FIELD OF THE INVENTION

The present invention relates to the cosmetic field, and, in particular, to an anti-ageing and revitalizing cosmetic gel composition having a high elasticizing, brightening and moisturising action on the skin.

### BACKGROUND OF THE INVENTION

A class of molecules still little used in the cosmetic sector for anti-ageing, elasticizing and moisturising purposes are peptides. Peptides, or rather oligopeptides, generally have a molecular weight of less than 5,000 daltons and consist of a chain of amino acids, which often “mimic” active parts of proteins normally found in the human body. For this reason, they are called “biomimetic” peptides, as they are able to bind to specific cell receptors and regulate biological processes. Each peptide works in very specific ways. In particular, when applied to the skin, peptides can perform specific functions, including stimulating, muscle relaxant, remodeling, repairing, strengthening, lightening, and desensitizing functions. Their use is usually recommended from the age of 35-40, depending on ageing state of the skin. The advantage of using peptides, in addition to their proven functionality, is their total safety of use as they faithfully mimic parts of molecules normally found in the human body. The possibilities of developing biomimetic peptides are countless, as well as the possibilities of creating of a mix of peptides with specific and complementary actions.

### SUMMARY OF THE INVENTION

The present invention provides a cosmetic gel composition for topical application to the skin, including an innovative and highly functional pool of biomimetic peptides for the well-being of the skin.

Further features and advantages of the cosmetic composition according to the present invention are also described.

As will be illustrated in detail below, owing to its particular combination of ingredients, as well as its pH and absorption characteristics, the cosmetic gel composition of the present invention has proved particularly effective in enhancing hydration and brightness of the skin, providing remarkable revitalizing, anti-ageing and regenerating effects.

### DETAILED DESCRIPTION

The cosmetic gel composition according to the present invention comprises the combination of the following components:

a peptide component, consisting of the combination of SH-Oligopeptide 2, SH-Polypeptide 1, SH-Oligopeptide 1 and SH-Polypeptide 42;

a gelling agent; and

water.

SH-Oligopeptide 2 is a single-chain recombinant human peptide having a maximum length of 153 amino acids and comprising the amino acid sequence SEQ ID NO:1. It can be produced by recombinant techniques known per se, including, for example, fermentation through a prokaryotic expression system, such as E. coli, by using a starting gene which is the synthetic copy of the human gene encoding for the Insulin-Like Growth Factor, used as such or adapted to the expression system used as the host. SH-Oligopeptide 2 contains a maximum of 153 amino acids and may include disulfide bridges and/or glycosylation.

SH-Polypeptide 1 is a single-chain recombinant human peptide having a maximum length of 155 amino acids and comprising the amino acid sequence SEQ ID NO:2. It can be produced by recombinant techniques known per se, including, for example, fermentation through a prokaryotic expression system, such as E. coli, by using a starting gene which is the synthetic copy of the human gene encoding for the Basic Fibroblast Growth Factor 2, used as such or adapted to the expression system used as the host. SH-Polypeptide 1 contains a maximum of 155 amino acids and may include disulfide bridges and/or glycosylation.

SH-Oligopeptide 1 is a single-chain recombinant human peptide having a maximum length of 53 amino acids and comprising the amino acid sequence SEQ ID NO:3. It can be produced by recombinant techniques known per se, including, for example, fermentation through a prokaryotic expression system, such as E. coli, by using a starting gene which is the synthetic copy of the human gene encoding for the Epidermal Growth Factor, used as such or adapted to the expression system used as the host. SH-Oligopeptide 1 contains a maximum of 53 amino acids and may include disulfide bridges and/or glycosylation.

SH-Polypeptide 42 is a single-chain recombinant human peptide having a maximum length of 177 amino acids and comprising the amino acid sequence SEQ ID NO:4. It can be produced by recombinant techniques known per se, including, for example, fermentation through a prokaryotic expression system, such as E. coli, by using a starting gene which is the synthetic copy of the human gene encoding for Interleukin-7, used as such or adapted to the expression system used as the host. SH-Polypeptide 42 contains a maximum of 177 amino acids and may include disulfide bridges and/or glycosylation.

SEQ ID NO: 1:
GPETLCGAEL VDALQFVCGD RGFYFNKPTG YGSSSRRAPQ
TGIVDECCFR SCDLRRLEMY CAPLKPAKSA
SEQ ID NO: 2:
AAGSITTLPA LPEDGGSGAF PPGHFKDPKR LYCKNGGFFL
RIHPDGRVDG VREKSDPHIK LQLQAEERGV VSIKGVCANR
YLAMKEDGRL LASKCVTDEC FFFERLESNN YNTYRSRKYT
SWYVALKRTG QYKLGSKTGP GQKAILFLPM SAKS
SEQ ID NO: 3:
MNSDSECPLS HDGYCLHDGV CMYIEALDKY ACNCVVGYIG
ERCQYRDLKW WELR
SEQ ID NO: 4:
DCDIEGKDGK QYESVLMVSI DQLLDSMKEI GSNCLNNEFN
FFKRHICDAN KEGMFLFRAA RKLRQFLKMN STGDFDLHLL
KVSEGTTILL NCTGQVKGRK PAALGEAQPT KSLEENKSLK
EQKKLNDLCF LKRLLQEIKT CWNKILMGTK EH

According to a preferred embodiment, SH-Oligopeptide 2, SH-Polypeptide 1, SH-Oligopeptide 1 and SH-Polypeptide 42 consist of the amino acid sequences SEQ ID Nos.: 1, 2, 3 and 4, respectively.

The gelling agent used in the cosmetic composition of the present invention is a rheology modifier capable of changing a mixture from the liquid to the gel phase. Gelling agents particularly suitable for use in the cosmetic composition of the present invention, which is water-based, are acrylic polymers and derivatives thereof, including carboxyvinyl polymers known under the trade name of Carbomer, which identifies a family of high-molecular-weight cross-linked acrylic acid homopolymers.

In a preferred embodiment, the cosmetic composition of the present invention also contains a hyaluronic acid salt, including high-molecular-weight sodium hyaluronate (preferably having a molecular weight greater than 1000 kDaltons, for example between 1000 and 5000 kDaltons). This substance has remarkable hygroscopic characteristics and contributes to improve skin barrier function and skin moisturization.

In another preferred embodiment, the cosmetic composition of the present invention also contains panthenol (provitamin B5). This molecule is a precursor of vitamin B5, which contributes to a filming, soothing and calming action on the skin; therefore, it can be considered a valid support for the regenerating activity of the skin cells performed by the oligopeptide combination described above.

According to further preferred embodiments, the cosmetic composition of the present invention also contains one or more of the following additional components: thickening agents, wetting agents, solubilizing agents, chelating agents, pH adjusting agents, and preserving agents.

An exemplary thickening agent suitable for use in the cosmetic composition of the present invention is xanthan gum, which is a high molecular weight polysaccharide with a thickening and stabilizing action on the “gel” form.

Exemplary wetting agents suitable for use in the cosmetic composition of the present invention are glycerin and propylene glycol. Propylene glycol is a so-called “skin enhancer” or “skin carrier”, as it enhances skin absorption not only of a product, but also of its main functional ingredients, namely the combination of oligopeptides described above. This ingredient may be replaced in the above formulation by propanediol, which, deriving from plants, is considered more skin friendly.

An exemplary solubilizing agent suitable for use in the cosmetic composition of the present invention is PEG-40 hydrogenated castor oil. This ingredient may or may not be used, if the degree of chemical-physical stability of the ingredients of the formulation and the risk of their separation is considered highly unlikely.

Exemplary preservatives suitable for use in the cosmetic composition of the present invention are dehydroacetic acid, benzoic acid, ethylhexylglycerin and phenoxyethanol, which are able to prevent any type of bacterial and/or fungal contamination of the formulation, particularly if used in a mixture.

An exemplary chelating agent suitable for use in the cosmetic composition of the present invention is ethylenediaminetetraacetic acid (EDTA) disodium salt, which is suitable in the preservative system by acting as a heavy metal chelator.

An exemplary pH adjusting agent suitable for use in the cosmetic composition of the present invention is sodium hydroxide, which is introduced in a quantity sufficient to adjust the pH of the formulation within the identified preferred range (pH between 5.00 and 5.70).

The preferred embodiments described above can be combined with each other as needed, and the implementation of such combinations falls within the skills of the person skilled in the art.

The table below illustrates, by way of non-limiting example, suitable percentages (% w/w) of the aforementioned ingredients of a cosmetic composition falling within the scope of the present invention:

| Ingredient | % w/w |
|---|---|
| Water | 75-95 |
| Glycerin | 1-5 |
| Propylene glycol or Propanediol | 1-5 |
| Carbomer | 0.1-1 |
| Sodium hyaluronate | 0.1-1 |
| Xanthan gum | 0.1-1 |
| Panthenol | 0.1-1 |
| Phenoxyethanol | 0.1-1 |
| PEG-40 hydrogenated castor oil | 0-1 |
| SH-Oligopeptide 2 | 0.0000001-0.1 |
| SH-Polypeptide 1 | 0.0000001-0.1 |
| SH-Oligopeptide 1 | 0.0000001-0.1 |
| SH-Polypeptide 42 | 0.0000001-0.1 |
| Dehydroacetic acid | 0.001-0.1 |
| Benzoic acid | 0.001-0.1 |
| Ethylhexylglycerin | 0.001-0.1 |
| EDTA disodium salt | 0.001-0.1 |
| Sodium hydroxide | 0.01-0.1 |

### EXPERIMENTAL SECTION

A formulation designed with the ingredients described in Table 1 and falling within the quantitative ranges described therein was subjected to a moisturizing, elasticizing and brightening efficacy study on 20 volunteers aged between 35 and 55 with dry skin. The study was performed under the supervision of a specialist in Dermatology and Venereology and included the following assessments:

1) Elasticizing Activity

Treatment area: face in the area between the cheekbones and the cheeks

At T0 and T30 days from the application of the product, mechanical tensiometric skin elasticity and tonicity measurements were carried out by using Cutometer® MPA 580—Corage+Khazaka electronic GmbH/Reviscometer (a probe for assessing skin elasticity), as well as a subjective evaluation by means of a self-assessment questionnaire also including a numerical score (VAS—Visual Analogical Scale) and judgments.

2) Lightening/Brightening Efficacy

Treatment Area: Face in the Cheek Area

The effect was quantified by: reflection colorimetry using the Chromameter CR-200—Minolta instrument, parameter L (luminance, expressed as a percentage: 0 for black and 100 for white); at T0 and T30 days from the application of the product. In addition, an objective clinical evaluation of the effectiveness of enhancing the brightness of the face was carried out, and pictures normalized for the light and distance of the subjects were taken. In this case too, a subjective evaluation was carried out by means of a self-assessment questionnaire including a numerical score (VAS—Visual Analogical Scale) and judgments.

3) Moisturizing Activity

Treatment Area: The Forearm

Corneometric measurements were carried out in quadruplicate at T30, T60 and T180 minutes (instrument used Corneometer MPA 580 and CM 825, Multiprobe G.F. Secchi) in order to assess the moisturizing activity. Three areas were identified on the volar surface of the forearms: two were treated with the product and the related placebo (which provides for the total absence of the aforementioned peptide mixture) under study, the third untreated area was used as a negative control.

The results of clinical tests showed that the formulation object of the present invention caused:
an increase in skin brightness after 30 days (T30) of application of the product. In particular, the L* parameter in the treated area is increased by a value equal to 2.45%; this variation is statistically significant (p<0.05); and
an increase in average skin hydration 30 (T30 min), 60 (T60 min) and 180 (T180 min) minutes after applying the product. In particular, the hydration in the area treated with the product is increased by a value equal to 9.27%, a value equal to 14.37% and a value equal to 14.36%, respectively, 30 minutes, 60 minutes and 180 minutes after application; these variations are statistically significant (p<0.05). It should be considered that the hydration in the area treated with the placebo is increased by a value equal to 3.78%, a value equal to 4.15% and a value equal to 3.48%, respectively, 30 minutes, 60 minutes and 180 minutes after application of the placebo; these variations are not statistically significant (p>0.05). Hydration in the untreated area is decreased by a value equal to 1.41% after 30 minutes, by a value equal to 1.50% after 60 minutes and by a value equal to 1.17% after 180 minutes; these variations are not statistically significant (p>0.05).
The placebo used in the clinical study is the same formulation as described in Table 1, but without the peptide pool.

Clinical evaluation also showed a general improvement in skin brightness. Furthermore, the product was well tolerated and positively judged by most of the volunteers who participated in the study.

What is claimed is:

1. A cosmetic gel composition for topical application to the skin, the cosmetic gel composition comprising:
a peptide component, consisting of the combination of:
a single-chain recombinant human peptide having a maximum length of 153 amino acids and comprising the amino acid sequence SEQ ID NO:1 (SH-Oligopeptide 2),
a single-chain recombinant human peptide having a maximum length of 155 amino acids and comprising the amino acid sequence SEQ ID NO:2 (SH-Polypeptide 1),
a single-chain recombinant human peptide having a maximum length of 53 amino acids and comprising the amino acid sequence SEQ ID NO:3 (SH-Oligopeptide 1), and
a single-chain recombinant human peptide having a maximum length of 177 amino acids and comprising the amino acid sequence SEQ ID NO:4 (SH-Polypeptide 42);
a gelling agent; and
water.

2. The cosmetic gel composition of claim 1, wherein the gelling agent is a carboxyvinyl polymer.

3. The cosmetic gel composition of claim 1, further comprising a high molecular weight hyaluronic acid salt.

4. The cosmetic gel composition of claim 3, further comprising panthenol.

5. The cosmetic gel composition of claim 1, further comprising one or more ingredients selected from the group consisting of: thickening agents, wetting agents, solubilizing agents, chelating agents, pH adjusting agents, preserving agents, and combinations thereof.

6. The cosmetic gel composition of claim 5, wherein the thickening agents comprise xanthan gum.

7. The cosmetic gel composition of claim 5, wherein the wetting agents comprise glycerine, propylene glycol and propanediol.

8. The cosmetic gel composition of claim 5, wherein the solubilizing agents comprise PEG-40 hydrogenated castor oil.

9. The cosmetic gel composition of claim 5, wherein the preservative agents comprise dehydroacetic acid, benzoic acid, ethylhexylglycerin and phenoxyethanol.

10. The cosmetic gel composition of claim 5, wherein the chelating agents comprise ethylenediaminetetraacetic acid (EDTA) disodium salt.

11. The cosmetic gel composition of claim 5, wherein the pH adjusting agents comprise sodium hydroxide.

12. The cosmetic gel composition of claim 1, having a pH ranging between 5.00 and 5.70.

13. The cosmetic gel composition of claim 1, wherein the cosmetic gel composition has skin moisturizing, brightening and/or revitalizing effects.


SEQUENCE LISTING (4 sequences)

SEQ ID NO: 1 - protein, 70 aa, Homo sapiens
GPETLCGAELVDALQFVCGDRGFYFNKPTGYGSSSRRAPQTGIVDECCFRSCDLRRLEMYCAPLKPAKSA

SEQ ID NO: 2 - protein, 154 aa, Homo sapiens
AAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 3 - protein, 54 aa, Homo sapiens
MNSDSECPLSHDGYCLHDGVCMYIEALDKYACNCVVGYIGERCQYRDLKWWELR

SEQ ID NO: 4 - protein, 152 aa, Homo sapiens
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH